(19) United States
(12) Patent Application Publication
(10) Pub. No.: US 2018/0217027 A1
(43) Pub. Date: Aug. 2, 2018

(54) CONCENTRATION CALCULATION APPARATUS AND GAS DETECTION APPARATUS

(71) Applicant: NGK Spark Plug Co., LTD., Nagoya (JP)

(72) Inventor: Yoshihiro NAKANO, Komaki (JP)

(73) Assignee: NGK Spark Plug Co., LTD., Nagoya (JP)

(21) Appl. No.: 15/872,432

(22) Filed: Jan. 16, 2018

(30) Foreign Application Priority Data
Jan. 19, 2017 (JP) 2017-007624
Jan. 10, 2018 (JP) 2018-001931

(51) Int. Cl. G01M 15/10; G01N 33/00; F01N 11/00; G01N 27/419

(52) U.S. Cl. CPC G01M 15/104; G01N 27/419; F01N 11/007; G01N 33/0037

(57) ABSTRACT

A multi-gas detection apparatus includes a multi-gas sensor and a control section. A microcomputer of the control section calculates the concentration of NOx by using a correction expression; i.e., by multiplying an NOx concentration output (═C) by a correction coefficient a and adding a correction addition value b. As shown in an expression, the value of the correction coefficient a is changed in accordance with the concentration of ammonia. As shown in an expression, the value of the correction addition value b is changed in accordance with the concentration of ammonia and the concentration of nitrogen dioxide. Even when a second pumping current Ip2 (in other words, the NOx concentration output) changes due to the influence of ammonia contained in the exhaust gas, the control section can compute the concentration of nitrogen oxide while mitigating the influence of ammonia.

This application claims the benefit of Japanese Patent Applications No. 2017-007624 filed Jan. 19, 2017 and No. 2018-001931 filed Jan. 10, 2018, all of which are incorporated herein by reference in their entirety.

### FIELD OF THE INVENTION

The present disclosure relates to a concentration calculation apparatus and a gas detection apparatus.

### BACKGROUND OF THE INVENTION

There has been known a concentration calculation apparatus which calculates the concentrations of ammonia (NH₃), nitrogen dioxide (NO₂), and nitrogen oxide (NOx) by using an NOx detection section which detects the concentration of nitrogen oxide and an ammonia detection section which detects the concentration of ammonia (Japanese Patent Application Laid-Open (kokai) No. 2015-34814).

In this concentration calculation apparatus, the ammonia concentration (NH₃ concentration), the nitrogen dioxide concentration (NO₂ concentration), and the nitrogen oxide concentration (NOx concentration) are calculated by using an NOx detection value provided by the NOx detection section, an ammonia detection value provided by the ammonia detection section, and a predetermined computation expression.

The concentration calculation apparatus can constitute a gas detection apparatus together with the NOx detection section and the ammonia detection section.

### Problems to be Solved by the Invention

However, in the above-described conventional concentration calculation apparatus, when the NOx detection value changes due to the influence of ammonia in a gas under measurement (target gas), the NOx detection value enters a state in which the NOx detection value correctly represents the concentration of nitrogen oxide, so that the accuracy in detecting the nitrogen oxide concentration may deteriorate.

More specifically, in a high temperature environment, ammonia and NOx gas react as represented by a formula NH₃+NOx=N₂+H₂O. For example, in some cases, NOx in the gas under measurement having flowed into a gas measurement chamber of a gas sensor element reacts with NH₃ in the gas under measurement having flowed into the gas measurement chamber. When NOx reacts with NH₃ as described above, the NOx concentration within the gas measurement chamber itself changes (decreases) as compared with the respective gas concentrations in the gas under measurement, and the NOx detection value also changes with a change in the NOx concentration. As a result, the accuracy in detecting the NOx concentration in the gas under measurement may deteriorate.

An object of the present disclosure is to provide a concentration calculation apparatus and a gas detection apparatus which can restrain deterioration of the accuracy in detecting the concentration of nitrogen oxide.

### SUMMARY OF THE INVENTION

### Means for Solving the Problems

One mode of the present disclosure is a concentration calculation apparatus which comprises a nitrogen oxide concentration computation section that calculates the concentration of nitrogen oxide contained in a target gas. The concentration calculation apparatus is adapted to be connected to a first detection section and a second detection section.

The first detection section is configured to output a first detection value which changes with the concentration of nitrogen oxide contained in the target gas. The second detection section is configured to output a second detection value which changes with the concentration of ammonia contained in the target gas.

The nitrogen oxide concentration computation section is configured to compute the concentration of the nitrogen oxide based on the first detection value.

The nitrogen oxide concentration computation section computes the concentration of the nitrogen oxide from a value obtained by multiplying the first detection value by a correction coefficient. The value of the correction coefficient is set based on the second detection value.

Even when the first detection value changes due to the influence of ammonia contained in the target gas, such a concentration calculation apparatus can compute the concentration of nitrogen oxide, while mitigating the influence of ammonia, by computing the concentration of nitrogen oxide through use of not only the first detection value but also the correction coefficient.

In particular, since the correction coefficient is set on the basis of the second detection value which changes with at least the concentration of ammonia, the concentration of nitrogen oxide can be computed in accordance with the concentration of ammonia contained in the target gas, whereby deterioration of the accuracy in detecting the nitrogen oxide concentration can be restrained.

Therefore, according to this concentration calculation apparatus, the influence of ammonia can be restrained at the time of detection of the concentration of nitrogen oxide contained in the target gas. Therefore, deterioration of the accuracy in detecting the nitrogen oxide concentration can be restrained.

Also, in the present disclosure, the nitrogen oxide concentration computation section may compute, as the concentration of the nitrogen oxide, a value obtained by multiplying the first detection value by the correction coefficient and adding a correction addition value to a result of the multiplication. The correction addition value is set based on at least the second detection value.

Even when the first detection value changes due to the influence of ammonia contained in the target gas, such a concentration calculation apparatus can compute the concentration of nitrogen oxide, while mitigating the influence of ammonia, by computing the concentration of nitrogen oxide through use of not only the first detection value but also the correction coefficient and the correction addition value. In particular, since the correction coefficient and the correction addition value are set based on the second detection value which changes with at least the concentration of ammonia, the concentration of nitrogen oxide can be computed in accordance with the concentration of ammonia contained in the target gas, whereby deterioration of the accuracy in detecting the nitrogen oxide concentration can be restrained.

Notably, the first detection section may be configured to include a first pumping cell through which first pumping current flows as a result of pumping out of oxygen in the target gas introduced into a measurement chamber or pumping oxygen into the measurement chamber, the value of the first pumping current changing with the oxygen concentration in the target gas, and a second pumping cell through which second pumping current flows, the value of the second pumping current changing in accordance with the concentration of nitrogen oxide contained in the gas under measurement whose oxygen concentration has been adjusted in the measurement chamber by the first pumping cell. In this case, the second pumping current can be utilized as the first detection value.

Next, in the concentration calculation apparatus of the present disclosure, an ammonia concentration computation section which computes the concentration of the ammonia based on the second detection value may be provided. Such a concentration calculation apparatus is used as a concentration calculation apparatus for detecting the concentrations of nitrogen oxide and ammonia in the target gas.

Next, in the concentration calculation apparatus of the present disclosure, the second detection section may output, as the second detection value, the second detection value which changes with the concentration of ammonia contained in the target gas as well as the concentration of nitrogen dioxide contained in the target gas. Also, the concentration calculation apparatus of the present disclosure may comprise a nitrogen dioxide concentration computation section which computes the concentration of the nitrogen dioxide based on the second detection value.

In such a concentration calculation apparatus, since the correction addition value is set based on the second detection value which changes with at least the concentration of ammonia and the concentration of nitrogen dioxide, the concentration of nitrogen oxide can be computed in consideration of not only the concentration of ammonia contained in the target gas but also the concentration of nitrogen dioxide contained in the target gas. As a result, it is possible to compute the concentration of nitrogen oxide while suppressing the influences of the concentration of ammonia and the concentration of nitrogen dioxide. Therefore, deterioration of the accuracy in detecting the nitrogen oxide concentration can be restrained.

Next, in the concentration calculation apparatus of the present disclosure, the first detection section and the second detection section are are integrally formed to provide a multi-gas sensor.

Since the multi-gas sensor includes the first detection section and the second detection section in an integrated form, it is utilized for an application in which the concentrations of nitrogen oxide, ammonia, and nitrogen dioxide which are contained in the same target gas are detected.

Therefore, according to this concentration calculation apparatus, deterioration of the accuracy in detecting the nitrogen oxide concentration can be restrained when using the multi-gas sensor for detecting the concentrations of nitrogen oxide, ammonia, and nitrogen dioxide in the target gas.

Notably, the second detection value used for changing the correction coefficient and the correction addition value is not limited to the second detection value itself, and an arbitrary value may be used as the second detection value, so long as the arbitrary value changes with the concentration of ammonia. For example, in the case where the second detection value changes with the ammonia concentration in the target gas, the correction coefficient and the correction addition value may be set in accordance with the ammonia concentration computed based on the second detection value. Alternatively, in the case where the second detection value changes with the ammonia concentration and the nitrogen dioxide concentration in the target gas under measurement, the correction coefficient and the correction addition value may be set in accordance with the ammonia concentration and the nitrogen dioxide concentration computed based on the second detection value. Notably, the first detection value used when the nitrogen oxide concentration is obtained is not limited to the first detection value itself, and an arbitrary value may be used as the first detection value, so long as the arbitrary value changes with the concentration of nitrogen oxide.

Next, another mode of the present disclosure is a gas detection apparatus which includes a first detection section, a second detection section, and the above-described concentration calculation apparatus.

Since this gas detection apparatus includes the above-described concentration calculation apparatus, the gas detection apparatus can suppress the influence of ammonia at the time of detection of the concentration of nitrogen oxide contained in the gas under measurement. Therefore, the gas detection apparatus can restrain deterioration of the accuracy in detecting the nitrogen oxide concentration.

### BRIEF DESCRIPTION OF THE DRAWINGS

These and other features and advantages of the present invention will become more readily appreciated when considered in connection with the following detailed description and appended drawings, wherein like designations denote like elements in the various views, and wherein:

FIG. 1 is a cross-sectional view showing the internal structure of a multi-gas sensor 2.

FIG. 2 is an illustration showing the schematic configuration of a multi-gas detection apparatus 1.

FIG. 3 is a cross-sectional view showing the structures of a first ammonia detection section 102 and a second ammonia detection section 103.

FIG. 4 is an explanatory graph and table showing the results of calculation of NOx concentration output calculated through use of an NOx detection section 101 for the case where ammonia concentration is changed in five types of gases under measurement having different nitrogen monoxide concentrations (NO concentrations).

FIG. 5 is an explanatory graph and table showing the results of calculation of NOx concentration output calculated through use of the NOx detection section for the case where ammonia concentration is changed in five types of gases under measurement having different nitrogen dioxide concentrations (NO₂ concentrations).

FIG. 6 is an explanatory graph and table showing NOx concentration versus “ammonia (NH₃)/nitrogen oxide (NOx) relative sensitivity ratio.”

FIG. 7 is an explanatory graph and table showing NOx concentration versus “ammonia (NH₃)/nitrogen oxide (NOx) relative sensitivity ratio minus change amount.”

### DETAILED DESCRIPTION OF THE INVENTION

Below, embodiments to which the present invention is applied will be described with reference to the drawings. Notably the present invention is not limited to the following embodiments, and the present invention may be practiced in various forms so long as they fall within the technical scope of the present invention.

### 1. First Embodiment

### 1-1. Overall Configuration

A multi-gas detection apparatus 1 which is provided for an internal combustion engine of an automobile or the like will be described as a first embodiment.

The multi-gas detection apparatus 1 is used for a urea SCR system which is mounted on a vehicle and which cleans nitrogen oxide contained in exhaust gas discharged from a diesel engine. More specifically, the multi-gas detection apparatus 1 detects the concentrations of ammonia, nitrogen dioxide, and nitrogen oxide contained in the exhaust gas. In the following description, the vehicle on which the multi-gas detection apparatus 1 is mounted is referred to as the own vehicle. Nitrogen dioxide and nitrogen oxide are denoted as NO₂ and NOx, respectively. Also, SCR is an abbreviation for selective catalytic reduction.

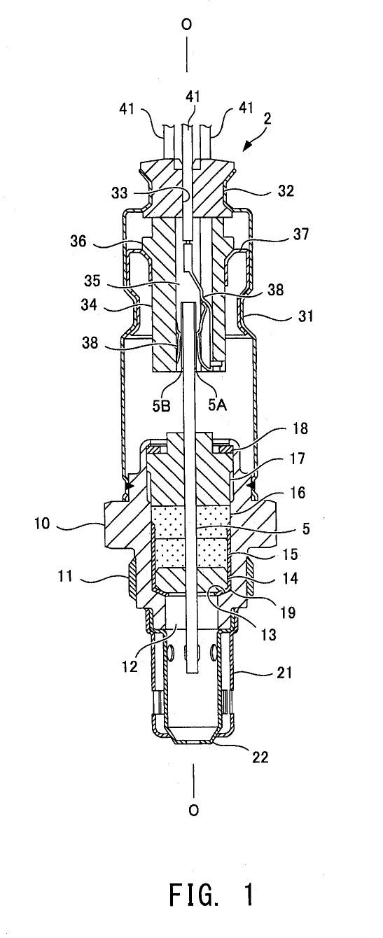

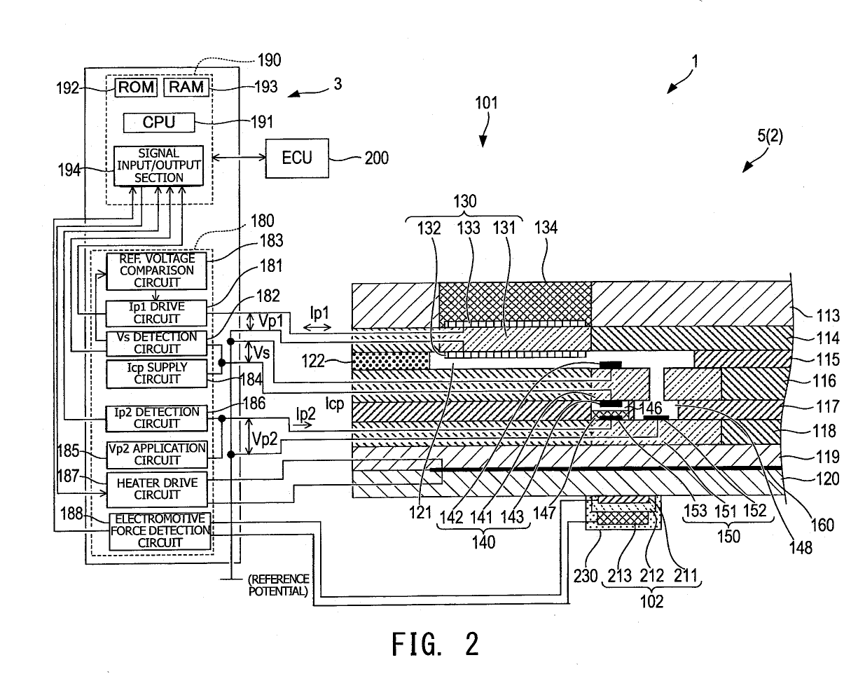

The multi-gas detection apparatus 1 includes a multi-gas sensor 2 shown in FIG. 1 and a control section 3 shown in FIG. 2.

The multi-gas sensor 2 is configured to detect NO₂ concentration, NOx concentration, and ammonia concentration (NH₃ concentration).

As shown in FIG. 2, the control section 3 is electrically connected to an electronic control apparatus 200 (also referred to as ECU 200) mounted on the own vehicle. The electronic control apparatus 200 receives data representing the NO₂ concentration, NOx concentration, and ammonia concentration (NH₃ concentration) in exhaust gas calculated in the control section 3, and executes, on the basis of the received data, a process of controlling the operation state of the diesel engine and a process of cleaning NOx accumulated in a catalyst.

### 1-2. Multi-Gas Sensor

As shown in FIG. 1, the multi-gas sensor 2 includes a sensor element section 5, a metallic shell 10, a separator 34, and connection terminals 38. In the following description, the side of the multi-gas sensor 2 on which the sensor element section 5 is disposed (i.e., the lower side in FIG. 1) is referred to as a forward end side, and the side on which the connection terminals 38 are disposed (i.e., the upper side in FIG. 1) is referred to as a rear end side.

The sensor element section 5 has a plate shape extending in the direction of an axial line O. Electrode terminal portions 5A and 5B are disposed at the rear end of the sensor element section 5. In FIG. 1, only the electrode terminal portions 5A and 5B are shown as electrode terminal portions formed in the sensor element section 5 for the purpose of simplifying the drawing. However, in actuality, a plurality of electrode terminal portions are formed according to the number of, for example, electrodes of an NOx detection section 101, a first ammonia detection section 102, and a second ammonia detection section 103 described later.

The metallic shell 10 is a tubular member, and a threaded portion 11 used to fix the multi-gas sensor 2 to an exhaust pipe of the diesel engine is formed on the external surface of the metallic shell 10. The metallic shell 10 has a through hole 12 extending in the direction of the axial line O and a ledge 13 protruding inward in the radial direction of the through hole 12. The ledge 13 is formed as an inward tapered surface extending from the radially outer side of the through hole 12 toward its center and inclined toward the forward end side.

The metallic shell 10 holds the sensor element section 5 such that a forward end portion of the sensor element section 5 protrudes forward from the through hole 12 and a rear end portion of the sensor element section 5 protrudes rearward from the through hole 12.

A ceramic holder 14 that is a tubular member surrounding the radial circumference of the sensor element section 5, talc rings 15 and 16 that are layers of charged talc powder, and a ceramic sleeve 17 are stacked in this order inside the through hole 12 of the metallic shell 10 from the forward end side toward the rear end side.

A crimp packing 18 is disposed between the ceramic sleeve 17 and a rear end portion of the metallic shell 10. A metallic holder 19 is disposed between the ceramic holder 14 and the ledge 13 of the metallic shell 10. The talc ring 15 and the ceramic holder 14 are accommodated in the metallic holder 19, and as a result of compressive charging of the material of the talc ring 15, the metallic holder 19 and the talc ring 15 are hermetically integrated together. A rear end portion of the metallic shell 10 is crimped so as to press the ceramic sleeve 17 toward the forward end side through the crimp packing 18. Since the material of the talc ring 16 is compressively charged into the metallic shell 10, the hermetic seal between the inner circumferential surface of the metallic shell 10 and the outer circumferential surface of the sensor element section 5 is ensured.

An outer protector 21 with gas flow holes and an inner protector 22 with gas flow holes are provided at a forward end portion of the metallic shell 10. The outer protector 21 and the inner protector 22 are each a tubular member formed from a metallic material such as stainless steel and having a closed forward end. The inner protector 22 is welded to the metallic shell 10 in a state it covers a forward end portion of the sensor element section 5, and the outer protector 21 is welded to the metallic shell 10 in a state it covers the inner protector 22.

A forward end portion of an outer tube 31 formed into a tubular shape is fixed, by means of welding, to a rear outer circumference of the metallic shell 10. A grommet 32 is disposed in a rear end opening of the outer tube 31 so as to close the opening.

Lead wire insertion holes 33 into which lead wires 41 are inserted are formed in the grommet 32. The lead wires 41 are electrically connected to the electrode terminal portions 5A and 5B of the sensor element section 5.

The separator 34 is a tubular member disposed on the rear end side of the sensor element section 5. A space formed inside the separator 34 is an insertion hole 35 extending through the separator 34 in the direction of the axial line O. A flange portion 36 protruding radially outward is formed on the outer surface of the separator 34.

A rear end portion of the sensor element section 5 is inserted into the insertion hole 35 of the separator 34, and the electrode terminal portions 5A and 5B are disposed inside the separator 34.

A holding member 37 formed of metal and having a tubular shape is disposed between the separator 34 and the outer tube 31. The holding member 37 is in contact with the flange portion 36 of the separator 34 and also with the inner surface of the outer tube 31 and thereby holds the separator 34 such that the separator 34 is fixed to the outer tube 31.

The connection terminals 38 are members disposed inside the insertion hole 35 of the separator 34 and are electrically conductive members that electrically connect the electrode terminal portions 5A and 5B of the sensor element section 5 to their respective lead wires 41. In FIG. 1, only two connection terminals 38 are shown for the purpose of simplifying the illustration.

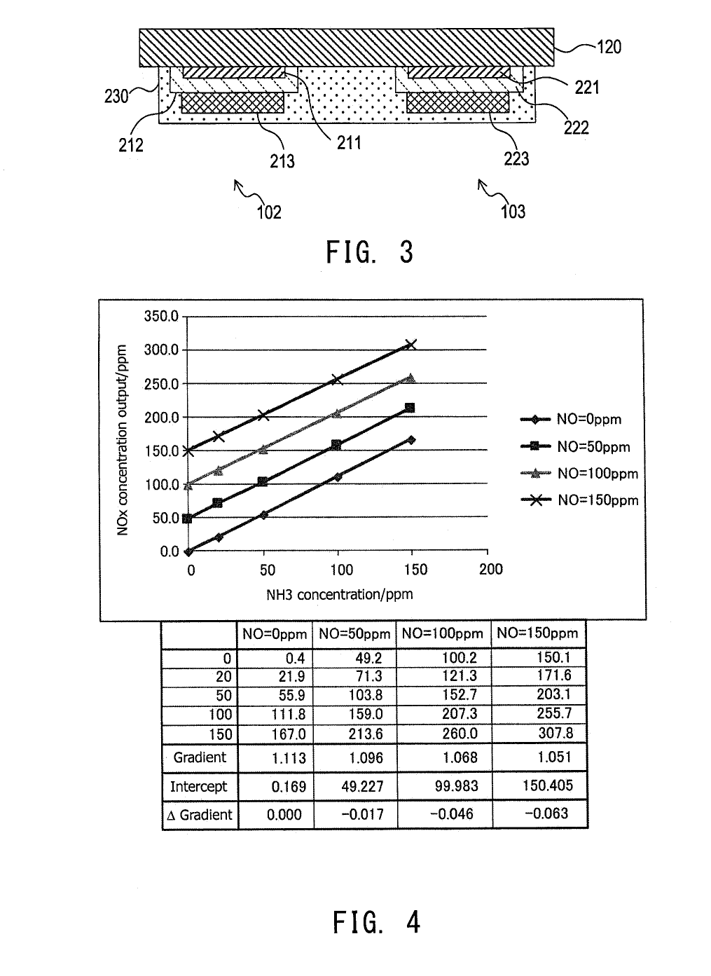

The sensor element section 5 includes the NOx detection section 101, the first ammonia detection section 102, and the second ammonia detection section 103. The second ammonia detection section 103 is not shown in FIG. 2 but is shown in FIG. 3. The first ammonia detection section 102 and the second ammonia detection section 103 are disposed in parallel at approximately the same position as a reference electrode 143 in the longitudinal direction of the NOx detection section 101 (i.e., the left/right direction in FIG. 2) in such a manner that the first ammonia detection section 102 and the second ammonia detection section 103 are located at different positions in the width direction of the NOx detection section 101 (i.e., the direction perpendicular to the sheet on which FIG. 2 is depicted). Therefore, in FIG. 2, of the first ammonia detection section 102 and the second ammonia detection section 103, only the first ammonia detection section 102 is shown.

The NOx detection section 101 is formed by sequentially stacking an insulating layer 113, a ceramic layer 114, an insulating layer 115, a ceramic layer 116, an insulating layer 117, a ceramic layer 118, an insulating layer 119, and an insulating layer 120. The insulating layers 113, 115, 117, 119, and 120 and the ceramic layers 114, 116, and 118 are formed mainly of alumina.

The NOx detection section 101 includes a first measurement chamber 121 formed between the ceramic layer 114 and the ceramic layer 116. The NOx detection section 101 introduces the exhaust gas from the outside into the interior of the first measurement chamber 121 through a diffusion resistor 122 that is disposed between the ceramic layer 114 and the ceramic layer 116 so as to be adjacent to the first measurement chamber 121. The diffusion resistor 122 is formed of a porous material such as alumina.

The NOx detection section 101 includes a first pumping cell 130. The first pumping cell 130 includes a solid electrolyte layer 131 and pumping electrodes 132 and 133.

The solid electrolyte layer 131 is formed mainly of zirconia having oxygen ion conductivity. A part of the ceramic layer 114 is removed from a region in contact with the first measurement chamber 121. Instead of the ceramic layer 114, the solid electrolyte layer 131 is charged (embedded) in the resulting space.

The pumping electrodes 132 and 133 are formed mainly of platinum. The pumping electrode 132 is disposed on a surface of the solid electrolyte layer 131, which surface is in contact with the first measurement chamber 121. The pumping electrode 133 is disposed a surface of the solid electrolyte layer 131 on the side opposite the pumping electrode 132 with respect to the solid electrolyte layer 131. The insulating layer 113 is removed from a region in which the pumping electrode 133 is disposed and from a region around the pumping electrode 133, and the resulting space is filled with a porous material 134 instead of the insulating layer 113. The porous material 134 allows gas (e.g., oxygen) flow between the pumping electrode 133 and the outside.

The NOx detection section 101 includes an oxygen concentration detection cell 140. The oxygen concentration detection cell 140 includes a solid electrolyte layer 141, a detection electrode 142, and the reference electrode 143.

The solid electrolyte layer 141 is formed mainly of zirconia having oxygen ion conductivity. A part of the ceramic layer 116 is removed from a region on the rear end side (i.e., the right side of FIG. 2) of the solid electrolyte layer 131. Instead of the ceramic layer 116, the solid electrolyte layer 141 is charged (embedded) in the resulting space.

The detection electrode 142 and the reference electrode 143 are formed mainly of platinum. The detection electrode 142 is disposed on a surface of the solid electrolyte layer 141, which surface is in contact with the first measurement chamber 121. The reference electrode 143 is disposed on a surface of the solid electrolyte layer 141 on the side opposite the detection electrode 142 with respect to the solid electrolyte layer 141.

The NOx detection section 101 includes a reference oxygen chamber 146. The reference oxygen chamber 146 is a through hole formed by removing the insulating layer 117 from a region in which the reference electrode 143 is disposed and from a region around the reference electrode 143.

The NOx detection section 101 includes a second measurement chamber 148 disposed downstream of the first measurement chamber 121. The second measurement chamber 148 is formed rearward of the detection electrode 142 and the reference electrode 143 so as to penetrate through the solid electrolyte layer 141 and the insulating layer 117. The NOx detection section 101 introduces the exhaust gas discharged from the first measurement chamber 121 into the second measurement chamber 148.

The NOx detection section 101 includes a second pumping cell 150. The second pumping cell 150 includes a solid electrolyte layer 151 and pumping electrodes 152 and 153.

The solid electrolyte layer 151 is formed mainly of zirconia having oxygen ion conductivity. The ceramic layer 118 is removed from a region in contact with the reference oxygen chamber 146 and the second measurement chamber 148 and a region around this region. Instead of the ceramic layer 118, the solid electrolyte layer 151 is charged (embedded) in the resulting space.

The pumping electrodes 152 and 153 are formed mainly of platinum. The pumping electrode 152 is disposed on a surface of the solid electrolyte layer 151, which surface is in contact with the second measurement chamber 148. The pumping electrode 153 is disposed on a surface of the solid electrolyte layer 151 on the side opposite the reference electrode 143 with respect to the reference oxygen chamber 146. A porous material 147 is disposed inside the reference oxygen chamber 146 so as to cover the pumping electrode 153.

The NOx detection section 101 includes a heater 160. The heater 160 is a heat-generating resistor that is formed mainly of platinum and generates heat when energized and is disposed between the insulating layers 119 and 120.

The first ammonia detection section 102 is formed on the outer surface of the NOx detection section 101, more specifically on the insulating layer 120. The first ammonia detection section 102 is disposed at approximately the same position, with respect to the direction of the axial line O (i.e., the left/right direction in FIG. 2), as the reference electrode 143 in the NOx detection section 101.

The first ammonia detection section 102 includes a first reference electrode 211 formed on the insulating layer 120, a first solid electrolyte body 212 covering the front and side surfaces of the first reference electrode 211, and a first detection electrode 213 formed on the front surface of the first solid electrolyte body 212. Similarly, as shown in FIG. 3, the second ammonia detection section 103 includes a second reference electrode 221 formed on the insulating layer 120, a second solid electrolyte body 222 covering the front and side surfaces of the second reference electrode 221, and a second detection electrode 223 formed on the front surface of the second solid electrolyte body 222.

The first reference electrode 211 and the second reference electrode 221 are formed mainly of platinum used as an electrode material and more specifically formed of a material containing Pt and zirconium oxide. The first solid electrolyte body 212 and the second solid electrolyte body 222 are formed of an oxygen ion-conductive material such as yttria-stabilized zirconia. The first detection electrode 213 and the second detection electrode 223 are formed mainly of gold used as an electrode material and more specifically formed of a material containing Au and zirconium oxide. Notably, the electrode materials of the first detection electrode 213 and the second detection electrode 223 are selected such that the first ammonia detection section 102 and the second ammonia detection section 103 differ from each other in terms of the ratio between the sensitivity to ammonia and the sensitivity to NOx.

Also, the first ammonia detection section 102 and the second ammonia detection section 103 are covered with a porous protecting layer 230. The protecting layer 230 prevents adhesion of a poisoning material to the first detection electrode 213 and the second detection electrode 223 and adjusts the diffusion rate of ammonia flowing from the outside into the first ammonia detection section 102 and the second ammonia detection section 103. As described above, the first ammonia detection section 102 and the second ammonia detection section 103 function as mixed potential sensing sections.

### 1-3. Control Section

As shown in FIG. 2, the control section 3 includes a control circuit 180 and a microcomputer 190.

The control circuit 180 is an analog circuit disposed on a circuit board. The control circuit 180 includes an Ip1 drive circuit 181, a Vs detection circuit 182, a reference voltage comparison circuit 183, an Icp supply circuit 184, a Vp2 application circuit 185, an Ip2 detection circuit 186, a heater drive circuit 187, and an electromotive force detection circuit 188.

The pumping electrode 132, the detection electrode 142, and the pumping electrode 152 are connected to a reference potential. The pumping electrode 133 is connected to the Ip1 drive circuit 181. The reference electrode 143 is connected to the Vs detection circuit 182 and the Icp supply circuit 184. The pumping electrode 153 is connected to the Vp2 application circuit 185 and the Ip2 detection circuit 186. The heater 160 is connected to the heater drive circuit 187.

The Ip1 drive circuit 181 applies a voltage Vp1 between the pumping electrode 132 and the pumping electrode 133 to supply a first pumping current Ip1 and detects the supplied first pumping current Ip1.

The Vs detection circuit 182 detects the voltage Vs between the detection electrode 142 and the reference electrode 143 and outputs the detection result to the reference voltage comparison circuit 183.

The reference voltage comparison circuit 183 compares a reference voltage (e.g., 425 mV) with the output from the Vs detection circuit 182 (i.e., the voltage Vs) and outputs the comparison result to the Ip1 drive circuit 181. The Ip1 drive circuit 181 controls the flow direction and magnitude of the first pumping current Ip1 such that the voltage Vs becomes equal to the reference voltage, and adjusts the concentration of oxygen in the first measurement chamber 121 to a prescribed value at which decomposition of NOx does not occur.

The Icp supply circuit 184 causes a weak current Icp to flow between the detection electrode 142 and the reference electrode 143. As a result, oxygen is fed from the first measurement chamber 121 to the reference oxygen chamber 146 through the solid electrolyte layer 141, whereby the concentration of oxygen in the reference oxygen chamber 146 is set to be a prescribed oxygen concentration serving as a reference.

The Vp2 application circuit 185 applies a constant voltage Vp2 (e.g., 450 mV) between the pumping electrode 152 and the pumping electrode 153. As a result, in the second measurement chamber 148, NOx is dissociated through the catalytic action of the pumping electrodes 152 and 153 of the second pumping cell 150. The oxygen ions obtained as a result of the dissociation migrate in the solid electrolyte layer 151 between the pumping electrode 152 and the pumping electrode 153, so that a second pumping current Ip2 flows. The Ip2 detection circuit 186 detects the second pumping current Ip2.

The heater drive circuit 187 applies a positive voltage for heater energization to one end of the heater 160, which is a heat-generating resistor, and applies a negative voltage for heater energization to the other end of the heater 160 to thereby drive the heater 160.

The electromotive force detection circuit 188 detects the electromotive force between the first reference electrode 211 and the first detection electrode 213 (hereinafter referred to as a first ammonia electromotive force) and the electromotive force between the second reference electrode 221 and the second detection electrode 223 (hereinafter referred to as a second ammonia electromotive force), and outputs signals representing the detection results to the microcomputer 190.

The microcomputer 190 includes a CPU 191, a ROM 192, a RAM 193, and a signal input/output section 194.

The CPU 191 executes a process for controlling the sensor element section 5 on the basis of a program stored in the ROM 192. The signal input/output section 194 is connected to the Ip1 drive circuit 181, the Vs detection circuit 182, the Ip2 detection circuit 186, the heater drive circuit 187, and the electromotive force detection circuit 188. The signal input/output section 194 converts the voltage values of analog signals from the Ip1 drive circuit 181, the Vs detection circuit 182, the Ip2 detection circuit 186, and the electromotive force detection circuit 188 to digital data and outputs the digital data to the CPU 191.

Also, the CPU 191 outputs a driving signal to the heater drive circuit 187 through the signal input/output section 194 so as to control the electric power supplied to the heater 160 by means of pulse width modulation such that the heater 160 reaches a target temperature. Notably, to control the electric power supplied to the heater 160, any known method may be used. Specifically, the impedance of a cell (e.g., the oxygen concentration detection cell 140) of the NOx detection section 101 is detected, and then the amount of the electric power supplied is controlled such that the impedance detected reaches a target value.

Also, the CPU 191 reads various data from the ROM 192 and performs various computation processes on the basis of the value of the first pumping current Ip1, the value of the second pumping current Ip2, the value of the first ammonia electromotive force, and the value of the second ammonia electromotive force.

The ROM 192 stores a “first ammonia electromotive force−first ammonia concentration output relational expression,” a “second ammonia electromotive force−second ammonia concentration output relational expression,” a “first pumping current−oxygen concentration relational expression,” a “second pumping current−NOx concentration output relational expression,” a “first ammonia concentration output & second ammonia concentration output & oxygen concentration−corrected ammonia concentration relational expression,” a “first ammonia concentration output & second ammonia concentration output & oxygen concentration−corrected NO₂ concentration relational expression,” and an “NOx concentration output & corrected ammonia concentration & corrected NO₂ concentration−corrected NOx concentration relational expression.”

Notably, the “first ammonia concentration output & second ammonia concentration output & oxygen concentration−corrected ammonia concentration relational expression” corresponds to a correction expression (1) described below. The “first ammonia concentration output & second ammonia concentration output & oxygen concentration−corrected NO₂ concentration relational expression” corresponds to a correction expression (2) described below. The “NOx concentration output & corrected ammonia concentration & corrected NO₂ concentration−corrected NOx concentration relational expression” corresponds to a correction expression (3) described below.

Also, the various data may be set in the form of predetermined relational expressions as described above or may be set in other forms (for example, tables) so long as various gas concentrations can be calculated from the outputs of the sensor. Alternatively, they may be values obtained through the use of a model gas whose gas concentration is known.

The “first ammonia electromotive force−first ammonia concentration output relational expression” and the “second ammonia electromotive force−second ammonia concentration output relational expression” are expressions representing the relation between the ammonia electromotive forces outputted from the first ammonia detection section 102 and the second ammonia detection section 103 and the ammonia concentration outputs. The “first ammonia electromotive force−first ammonia concentration output relational expression” and the “second ammonia electromotive force−second ammonia concentration output relational expression” are relational expressions which are determined in advance through use of a model gas whose ammonia gas concentration is known.

The “first pumping current−oxygen concentration relational expression” is an expression representing the relation between the first pumping current and the oxygen concentration (i.e., the O₂ concentration) in the exhaust gas. The “second pumping current−NOx concentration output relational expression” is an expression representing the relation between the second pumping current and the NOx concentration output. The “first pumping current−oxygen concentration relational expression” and the “second pumping current−NOx concentration output relational expression” are relational expressions which are determined in advance through use of a model gas whose oxygen concentration and NOx concentration are known.

The “first ammonia concentration output & second ammonia concentration output & oxygen concentration−corrected ammonia concentration relational expression” is an expression representing the relation between the first and second ammonia concentration outputs affected by the oxygen concentration, the ammonia concentration, and the NO₂ concentration and the corrected ammonia concentration from which the influences of the oxygen concentration and the NO₂ concentration have been removed. The “first ammonia concentration output & second ammonia concentration output & oxygen concentration−corrected NO₂ concentration relational expression” is an expression representing the relation between the first and second ammonia concentration outputs affected by the oxygen concentration, the ammonia concentration, and the NO₂ concentration and the corrected NO₂ concentration from which the influences of the oxygen concentration and the ammonia concentration have been removed. The “NOx concentration output & corrected ammonia concentration & corrected NO₂ concentration−corrected NOx concentration relational expression” is an expression representing the relation between the NOx concentration output affected by the ammonia concentration and the NO₂ concentration and the corrected NOx concentration from which the influences of the ammonia concentration and the NO₂ concentration have been removed.

Next, a description will next be given of a computation process for obtaining the NO₂ concentration, the NOx concentration, and the ammonia concentration from the first pumping current Ip1, the second pumping current Ip2, the first ammonia electromotive force, and the second ammonia electromotive force. This computation process is executed by the CPU 191 of the microcomputer 190.

When the first pumping current Ip1, the second pumping current Ip2, the first ammonia electromotive force, and the second ammonia electromotive force are inputted, the CPU 191 performs a computation process for obtaining the oxygen concentration, the NOx concentration output, the first ammonia concentration output, and the second ammonia concentration output. Specifically, the CPU 191 calls the “first ammonia electromotive force−first ammonia concentration output relational expression,” the “second ammonia electromotive force−second ammonia concentration output relational expression,” the “first pumping current Ip1−oxygen concentration relational expression,” and the “second pumping current Ip2−NOx concentration output relational expression” from the ROM 192 and then calculates the oxygen concentration and other concentration outputs using these relational expressions.

Notably, the “first ammonia electromotive force−first ammonia concentration output relational expression” and the “second ammonia electromotive force−second ammonia concentration output relational expression” are set such that, over the entire range of the ammonia electromotive forces outputted from the first and second ammonia detection sections 102 and 103 in their use environment, an approximately linear relation is present between each of the ammonia concentration outputs from the ammonia detection section and the ammonia concentration in the gas under measurement. Since these conversion expressions are used for conversion, in the correction expressions below, calculation which utilizes changes in gradient and offset is possible.

After the oxygen concentration, the NOx concentration output, the first ammonia concentration output, and the second ammonia concentration output are obtained, the CPU 191 performs computations using the correction expressions described below to obtain the ammonia concentration, NO₂ concentration, and NOx concentration in the exhaust gas.

x=F(A,B,D)=(eA−c)*(jB−h−fA+d)/(eA−c−iB+g)+fA−d Correction expression (1):

y=F′(A,B,D)=(jB−h−fA+d)/(eA−c−iB+g) Correction expression (2):

z=a*C+b Correction expression (3):

a=f(x)=1/(1−γx) Correction expression (4):

b=f′(x,y)=δxy/(1−γx)−βx/(1−γx)−αy/(1−γx)+y Correction expression (5):

In these correction expressions, x represents the ammonia concentration, y represents the NO₂ concentration, and z represents the NOx concentration. A represents the first ammonia concentration output, B represents the second ammonia concentration output, C represents the NOx concentration output, and D represents the oxygen concentration. F in the correction expression (1) represents that x is a function of (A, B, D), and F′ in the correction expression (2) represents that y is a function of (A, B, D). a and b are correction values (correction coefficient, correction addition value) and are correction values calculated through use of the ammonia concentration and the NO₂ concentration (namely, correction values determined by x and y). f of the expression (4) represents that a is a function of (x), and f′ of the expression (5) represents that b is a function of (x, y). α, β, γ, and δ are coefficients determined on the basis of the characteristics of the NOx detection section 101. c, d, e, f, g, h, i, and j are coefficients calculated by using the oxygen concentration D (i.e., coefficients determined by D).

Notably, the correction expressions (3) to (5) are determined on the basis of a relational expression (6) which represents the relation between the NOx concentration output (═C) and the concentrations of respective gases (ammonia concentration (=x), NO₂ concentration (=y), and NO concentration (=u)). A relational expression (7) which represents the NO concentration (=u) is obtained by modifying the expression (6). Also, the NOx concentration (z) is represented by a relational expression (8) in which the NO₂ concentration (=y) and the NO concentration (=u) are used. As can be understood from these, the above-described expressions (3) to (5) are obtained by substituting the expression (7) into the expression (8).

C=u+αy+βx−γxu−δxy=(1−γx)*u+αy+βx−δxy Relational expression (6):

u=(C+δxy−βx−αy)/(1−γx)=C/(1−γx)+δxy/(1−γx)−βx/(1−γx)−αy/(1−γx) Relational expression (7):

z=u+y Relational expression (8):

Notably, although the NOx detection section 101 has characteristics which allow the detection of nitrogen oxide (NOx) including nitrogen dioxide (NO₂) and nitrogen monoxide (NO), the NOx concentration output (═C) may change due to the influence of ammonia contained in the gas under measurement. In the case where such an influence is taken into consideration, the NOx concentration output (═C) can be represented by the above-described relational expression (6) through use of the ammonia concentration (=x), the nitrogen dioxide concentration (=y) and the nitrogen monoxide concentration (=u).

Therefore, use of the correction expressions (3) to (5) determined on the basis of the expression (6) makes it possible to obtain the NOx concentration (=z) while taking the influence of ammonia in the gas under measurement into consideration. The NOx concentration obtained in this manner has a reduced error caused by the influence of ammonia.

The CPU 191 obtains the ammonia concentration, the NO₂ concentration, and the NOx concentration in the exhaust gas by computing them through substitution of the first ammonia concentration output, the second ammonia concentration output, the NOx concentration output, and the oxygen concentration into the above-described correction expressions (1) to (5).

Notably, the correction expression (1) and the correction expression (2) are expressions determined on the basis of the characteristics of the first ammonia detection section 102 and the second ammonia detection section 103, and the correction expressions (3) to (5) are expressions determined on the basis of the characteristics of the NOx detection section 101. Also, the correction expressions (1) to (5) are merely examples of correction expressions, and other correction expressions, coefficients, etc. may be appropriately used in accordance with the gas detection characteristics. Also, the way of correction is not limited to a way in which the values are updated continuously and may be a way in which the values are updated discretely (stepwise). For example, the correction values may be updated in accordance with the above-described expressions in each processing sampling of the CPU or at predetermined intervals, and the update timing may be changed in accordance with the range of gas concentration.

Notably, the various functions of the microcomputer 190 are realized by a program which is stored in a non-transitory tangible recording medium and executed by the CPU. In this example, the ROM corresponds to the non-transitory tangible recording medium storing the program. Also, a method corresponding to the program is performed as a result of execution of the program. Notably, the control section 3 may include a single microcomputer or a plurality of microcomputers. Also, some or all of the functions of the microcomputer may be realized by hardware; for example, by a single IC or a plurality of ICs.

The multi-gas detection apparatus 1 including the control section 3 configured as described above calculates the concentrations of ammonia, NO₂, and NOx contained in the exhaust gas through use of the multi-gas sensor 2 which includes the NOx detection section 101, the first ammonia detection section 102, and the second ammonia detection section 103.

### 1-4. Characteristics of NOx Detection Section

Next, the characteristics of the NOx detection section 101 will be described.

The graph and table of FIG. 4 show the results of calculation of the NOx concentration output (═C) calculated through use of the NOx detection section 101 for the case where the ammonia concentration is changed in five types of gases under measurement having different nitrogen monoxide concentrations (NO concentrations (=u)). Notably, the “gradient and intercept” of each graph were calculated, and the “change amount of gradient (=Δ gradient)” was calculated with the gradient in the case where the nitrogen monoxide concentration (NO concentration) was 0 [ppm] being used as a reference. These calculated values are shown in the table.

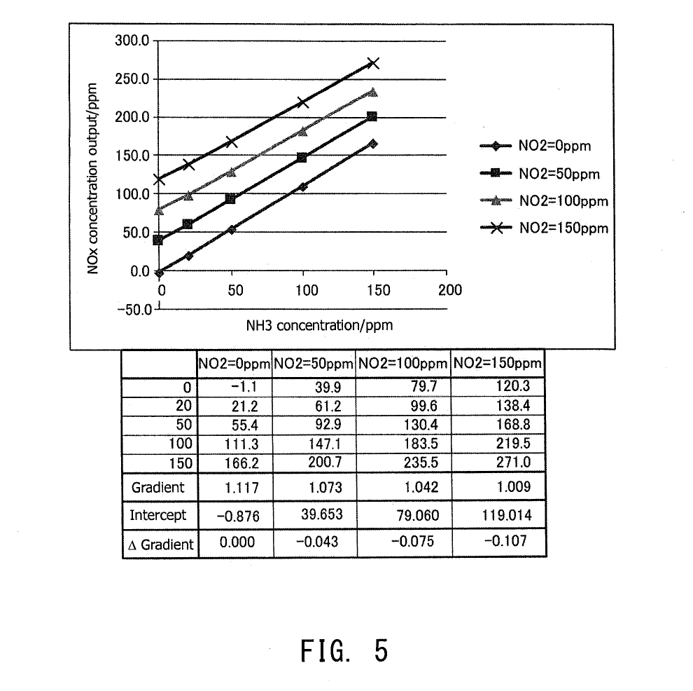

Similarly, the graph and table of FIG. 5 show the results of calculation of the NOx concentration output (═C) calculated through use of the NOx detection section 101 for the case where the ammonia concentration is changed in five types of gases under measurement having different nitrogen dioxide concentrations (NO₂ concentrations (=y)). Notably, the “gradient and intercept” of each graph were calculated, and the “change amount of gradient (=Δ gradient)” was calculated with the gradient in the case where the nitrogen dioxide concentration (NO₂ concentration) was 0 [ppm] being used as a reference. These calculated values are shown in the table.

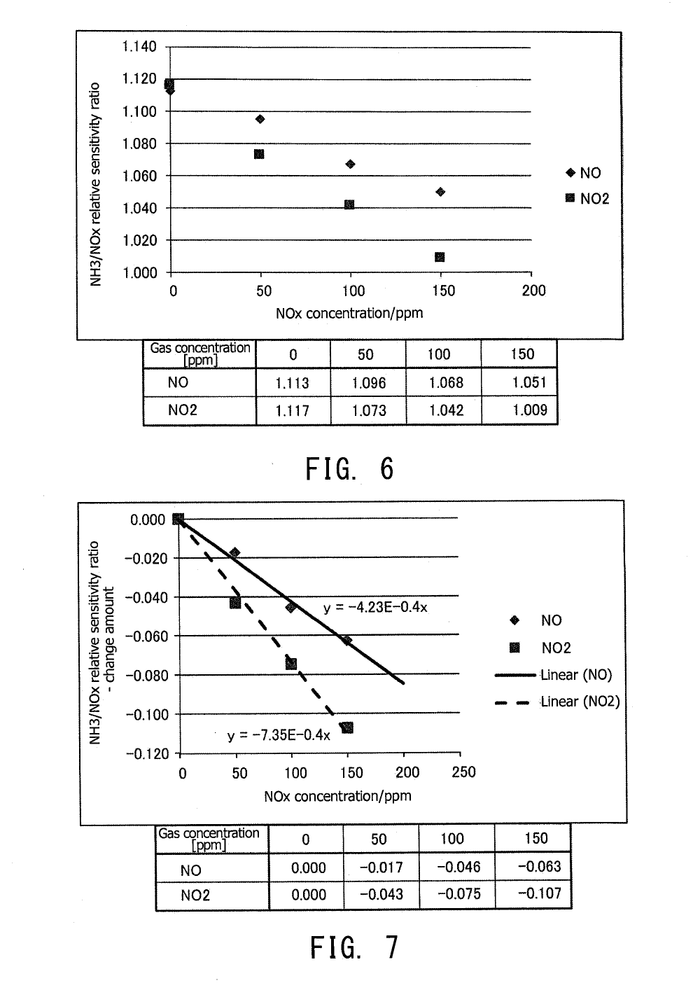

Also, FIG. 6 shows the NOx concentration versus the “ammonia (NH₃)/nitrogen oxide (NOx) relative sensitivity ratio” based on the above-described calculation results. Notably, since the relative sensitivity ratio assumes a value corresponding to the gradient of the above-described graph, FIG. 6 shows the correlation between the NOx concentration and the gradient of the nitrogen monoxide concentration (NO concentration) and the correlation between the NOx concentration and the gradient of the nitrogen dioxide concentration (NO₂ concentration).

FIG. 7 shows the NOx concentration versus the “ammonia (NH₃)/nitrogen oxide (NOx) relative sensitivity ratio minus change amount” based on the results shown in FIG. 6. Specifically, while the relative sensitivity ratio in the case where the NOx concentration is 0 [ppm] is used as a reference, “the difference of the relative sensitivity ratio” is calculated as the “ammonia (NH₃)/nitrogen oxide (NOx) relative sensitivity ratio minus change amount.”

As can be understood from the calculation results shown in FIG. 4 to FIG. 7, the NOx concentration output calculated through use of the NOx detection section 101 changes in value due to the influence of ammonia contained in the gas under measurement. As can be understood from the calculation results mentioned above, the degree of the influence of ammonia on the nitrogen monoxide concentration (NO concentration) differs from the degree of the influence of ammonia on the nitrogen dioxide concentration (NO₂ concentration).

Since the NOx detection section 101 has such characteristics, the NOx concentration output (═C) can be represented by the above-described expression (6).

Therefore, as described above, through use of the correction expressions (3) to (5) determined on the basis of the expression (6), it becomes possible to obtain the NOx concentration (=z) while taking the influence of ammonia contained in the gas under measurement into consideration. Thus, the NOx concentration has a reduced error caused by the influence of ammonia.

### 1-5. Effects

As described above, the multi-gas detection apparatus 1 of the present embodiment includes the multi-gas sensor 2 and the control section 3.

The multi-gas sensor 2 includes the sensor element section 5, and the sensor element section 5 includes the NOx detection section 101 and the ammonia detection section (the first ammonia detection section 102, the second ammonia detection section 103).

The control section 3 calculates the concentrations of ammonia, nitrogen dioxide, and nitrogen oxide contained in exhaust gas (gas under measurement) through use of the NOx detection section 101 and the ammonia detection section (the first ammonia detection section 102, the second ammonia detection section 103).

The NOx detection section 101 is configured to output the second pumping current Ip2 whose value changes with the concentration of nitrogen oxide (NOx) contained in the exhaust gas. Each of the first ammonia detection section 102 and the second ammonia detection section 103 is configured to output ammonia electromotive force whose value changes with the concentrations of ammonia and nitrogen dioxide contained in the exhaust gas.

The microcomputer 190 of the control section 3 computes, on the basis of the “second pumping current Ip2−NOx concentration output relational expression,” the NOx concentration output (═C) corresponding to the detected second pumping current Ip2.

Also, the microcomputer 190 of the control section 3 computes, on the basis of the “first ammonia electromotive force−first ammonia concentration output relational expression” and the “second ammonia electromotive force−second ammonia concentration output relational expression,” the first ammonia concentration output (=A) and the second ammonia concentration output (═B) which correspond to the ammonia electromotive forces output from the first ammonia detection section 102 and the second ammonia detection section 103, respectively.

Then, the microcomputer 190 computes the ammonia concentration (=x) on the basis of the obtained first and second ammonia concentration outputs and the correction expression (1). Also, the microcomputer 190 computes the nitrogen dioxide concentration (=y) on the basis of the obtained first and second ammonia concentration outputs and the correction expression (2).

Also, the microcomputer 190 computes the NOx concentration through use of the correction expression (3); i.e., computes, as the NOx concentration, a value obtained by multiplying the NOx concentration output (═C) by the correction coefficient a and adding the correction addition value b to the result of the multiplication. At that time, as shown in the expression (4), the value of the correction coefficient a is calculated on the basis of the ammonia concentration. Also, as shown in the expression (5), the value of the correction addition value b is calculated on the basis of the ammonia concentration and the nitrogen dioxide concentration.

Even when the second pumping current Ip2 (in other words, the NOx concentration output (═C)) changes due to the influence of ammonia contained in the exhaust gas, the control section 3 having such a configuration can compute the concentration of nitrogen oxide, while mitigating the influence of ammonia, by computing the concentration of nitrogen oxide through use of not only the NOx concentration output but also the correction coefficient a and the correction addition value b.

In particular, since the correction coefficient a and the correction addition value b are set (changed) on the basis of at least the concentration of ammonia, the concentration of nitrogen oxide can be computed in accordance with the concentration of ammonia contained in the exhaust gas, whereby deterioration of the accuracy in detecting the nitrogen oxide concentration can be restrained.

Therefore, since this control section 3 can suppress an error caused by the influence of ammonia at the time of detection of the concentration of nitrogen oxide contained in the exhaust gas, the control section 3 can restrain deterioration of the accuracy in detecting the nitrogen oxide concentration.

Also, in this control section 3, the correction addition value b is set (changed) on the basis of the concentration of ammonia and the concentration of nitrogen dioxide. Therefore, the concentration of nitrogen oxide can be computed in consideration of not only the concentration of ammonia contained in the exhaust gas but also the concentration of nitrogen dioxide contained in the exhaust gas.

As a result, the control section 3 can compute the concentration of nitrogen oxide while suppressing the influences of the concentration of ammonia and the concentration of nitrogen dioxide. Therefore, deterioration of the accuracy in detecting the nitrogen oxide concentration can be restrained.

Next, in the multi-gas detection apparatus 1, the NOx detection section 101 and the ammonia detection section (the first ammonia detection section 102, the second ammonia detection section 103) are provided as the multi-gas sensor 2 in which they are integrally formed.

Since such a multi-gas sensor 2 includes the NOx detection section 101 and the ammonia detection section (the first ammonia detection section 102, the second ammonia detection section 103) in an integrated form, it is utilized for an application in which the concentrations of nitrogen oxide, ammonia, and nitrogen dioxide which are contained in the same exhaust gas are detected.

Therefore, the multi-gas detection apparatus 1 can restrain deterioration of the accuracy in detecting the nitrogen oxide concentration in the case of use of the multi-gas sensor 2 for detecting the concentrations of nitrogen oxide, ammonia, and nitrogen dioxide in the exhaust gas.

Since the multi-gas detection apparatus 1 includes the control section 3, the multi-gas detection apparatus 1 can suppress an error caused by the influence of ammonia in detecting the concentration of nitrogen oxide contained in the exhaust gas. Therefore, the multi-gas detection apparatus 1 can restrain deterioration of the accuracy in detecting the nitrogen oxide concentration.

### 1-6. Correspondence Between Terms in Embodiment and Terms in Claims

A description will be given of the correspondence between terms used in the present embodiment and terms used in the claims.

The multi-gas detection apparatus 1 corresponds to the gas detection apparatus; the control section 3 corresponds to the concentration calculation apparatus; the NOx detection section 101 corresponds to the first detection section; and the ammonia detection section (the first ammonia detection section 102, the second ammonia detection section 103) corresponds to the second detection section.

The microcomputer 190 which computes the NOx concentration by using the expressions (3) to (5) corresponds to the nitrogen oxide concentration computation section; the microcomputer 190 which computes the ammonia concentration by using the expression (1) corresponds to the ammonia concentration computation section; and the microcomputer 190 which computes the nitrogen dioxide concentration by using the expression (2) corresponds to the nitrogen dioxide concentration computation section.

### 2. Second Embodiment

### 2-1. Overall Configuration

A multi-gas detection apparatus which computes the NOx concentration through use of correction expressions (9) and (10) instead of the correction expressions (3) to (5) in the first embodiment will be described as a second embodiment. Notably, the multi-gas detection apparatus of the second embodiment includes a control section and a multi-gas sensor similar to those of the first embodiment, and has the same hardware configuration as the first embodiment. In the following description regarding the second embodiment, the point different from the first embodiment will be mainly described.

### 2-2. Computation Process Executed at Control Section

There will be described a computation process of the second embodiment for obtaining the NO₂ concentration, the NOx concentration, and the ammonia concentration from the first pumping current Ip1, the second pumping current Ip2, the first ammonia electromotive force, and the second ammonia electromotive force. This computation process is executed by the CPU 191 of the microcomputer 190.

After obtainment of the oxygen concentration, the NOx concentration output, the first ammonia concentration output, and the second ammonia concentration output, the CPU 191 obtains the ammonia concentration and the NO₂ concentration in the exhaust gas by performing a computation in which the above-described correction expressions (1) and (2) are used, and obtains the NOx concentration in the exhaust gas by performing a computation in which the following correction expressions are used.

z=p*C+q Correction expression (9):

p=f″(x)=1/(1−mx) Correction expression (10):

q=f′″(x)=−kx/(1−mx) Correction expression (11):

In these correction expressions, x represents the ammonia concentration, and z represents the NOx concentration. C represents the NOx concentration output. p and q are correction values (correction coefficient, correction addition value) and are correction values calculated through use of the ammonia concentration (namely, the correction value determined by x). f″ of the expression (10) represents that p is a function of (x), and f′″ of the expression (11) represents that q is a function of (x). k and m are coefficients determined on the basis of the characteristics of the NOx detection section 101.

Notably, the correction expressions (9) to (11) are determined on the basis of a relational expression (12) which represents the relation between the NOx concentration output (═C) and the concentrations of respective gases (ammonia concentration (=x) and NOx concentration (=z)). A relational expression (13) which represents the NOx concentration (=z) is obtained by modifying the expression (12). The above-described expressions (9) to (11) are obtained on the basis of the expression (13) obtained on the basis of the expression (12).

C=z+kx−mxz=(1−mx)*z+kx Relational expression (12):

z=(C−kx)/(1−mx)=C/(1−mx)−kx/(1−mx) Relational expression (13):

Notably, although the NOx detection section 101 has characteristics which allow the detection of nitrogen oxide (NOx), the NOx concentration output (═C) may change due to the influence of ammonia contained in the gas under measurement. In the case where such an influence is taken into consideration, the NOx concentration output (═C) can be represented by the above-described relational expression (12) through use of the ammonia concentration (=x) and the nitrogen oxide concentration (=z).

Therefore, use of the correction expressions (9) to (11) determined on the basis of the expression (12) makes it possible to obtain the NOx concentration (=z) while taking the influence of ammonia in the gas under measurement into consideration. The NOx concentration obtained in this manner has a reduced error caused by the influence of ammonia.

The CPU 191 obtains the ammonia concentration, the NO₂ concentration, and the NOx concentration in the exhaust gas by computing them through substitution of the first ammonia concentration output, the second ammonia concentration output, the NOx concentration output, and the oxygen concentration into the above-described correction expressions (1), (2), and (9) to (11).

Notably, the correction expression (1) and the correction expression (2) are expressions determined on the basis of the characteristics of the first ammonia detection section 102 and the second ammonia detection section 103, and the correction expressions (9) to (11) are expressions determined on the basis of the characteristics of the NOx detection section 101. Also, the correction expressions (1), (2), and (9) to (11) are merely examples of correction expressions, and other correction expressions, coefficients, etc. may be appropriately used in accordance with the gas detection characteristics.

The multi-gas detection apparatus 1 including the control section 3 of the second embodiment configured as described above calculates the concentrations of ammonia, NO₂, and NOx contained in the exhaust gas through use of the multi-gas sensor 2 which includes the NOx detection section 101, the first ammonia detection section 102, and the second ammonia detection section 103.

### 2-3. Effects

As described above, the multi-gas detection apparatus 1 of the present second embodiment includes the multi-gas sensor 2 and the control section 3.

The microcomputer 190 of the control section 3 computes the ammonia concentration (=x) on the basis of the obtained first and second ammonia concentration outputs and the correction expression (1). Also, the microcomputer 190 computes the nitrogen dioxide concentration (=y) on the basis of the obtained first and second ammonia concentration outputs and the correction expression (2).

Also, the microcomputer 190 computes the NOx concentration through use of the correction expression (9); i.e., computes, as the NOx concentration, a value obtained by multiplying the NOx concentration output (═C) by the correction coefficient p and adding the correction addition value q to the result of the multiplication. At that time, as shown in the expression (10), the value of the correction coefficient p is calculated on the basis of the ammonia concentration. Also, as shown in the expression (11), the value of the correction addition value q is calculated on the basis of the ammonia concentration.

Even when the second pumping current Ip2 (in other words, the NOx concentration output (═C)) changes due to the influence of ammonia contained in the exhaust gas, the control section 3 having such a configuration can compute the concentration of nitrogen oxide, while mitigating the influence of ammonia, by computing the concentration of nitrogen oxide through use of not only the NOx concentration output but also the correction coefficient a and the correction addition value b.

In particular, since the correction coefficient p and the correction addition value q are set (changed) on the basis of at least the concentration of ammonia, the concentration of nitrogen oxide can be computed in accordance with the concentration of ammonia contained in the exhaust gas, whereby deterioration of the accuracy in detecting the nitrogen oxide concentration can be restrained.

Therefore, since this control section 3 can suppress an error caused by the influence of ammonia at the time of detection of the concentration of nitrogen oxide contained in the exhaust gas, the control section 3 can restrain deterioration of the accuracy in detecting the nitrogen oxide concentration.

Since the multi-gas detection apparatus 1 includes the control section 3, the multi-gas detection apparatus 1 can suppress an error caused by the influence of ammonia at the time of detection of the concentration of nitrogen oxide contained in the exhaust gas. Therefore, the multi-gas detection apparatus 1 can restrain deterioration of the accuracy in detecting the nitrogen oxide concentration.

### 2-4. Correspondence Between Terms in Embodiment and Terms in Claims

A description will be given of the correspondence between terms used in the present embodiment and terms used in the claims.

The microcomputer 190 which computes the NOx concentration by using the expressions (9) to (11) corresponds to the nitrogen oxide concentration computation section; the microcomputer 190 which computes the ammonia concentration by using the expression (1) corresponds to the ammonia concentration computation section; and the microcomputer 190 which computes the nitrogen dioxide concentration by using the expression (2) corresponds to the nitrogen dioxide concentration computation section.

### 3. Other Embodiments

While the embodiments of the present invention have been described, the present invention is not limited to the above-described embodiments. The present invention can be implemented in various forms without departing from the gist of the present invention.

For example, in the above-described embodiments, there has been described a form in which a multi-gas sensor in which an NOx detection section and an ammonia detection section are integrally formed is provided as a sensor. However, the present invention is not limited to such a form. Specifically, there can be employed a form in which a sensor including an NOx detection section and a sensor including an ammonia detection section are provided separately.

Also, in the above-described embodiments, there has been described a configuration in which two detection sections (the first ammonia detection section 102, the second ammonia detection section 103) are provided as an ammonia detection section. However, there can be employed a form in which an ammonia detection section composed of a single detection section is provided.

In the above-described embodiments, the correction coefficient and the correction addition value are calculated from the ammonia concentration obtained from the second pumping current Ip2 and a predetermined relational expression. However, the calculation method is not limited to the method in which a relational expression is used so long as the correction coefficient and the correction addition value are set on the basis of the second pumping current Ip2. For example, the relation between the second pumping current Ip2 and “the correction coefficient and the correction addition value” may be set as a table. Also, in the above-described embodiments, the NOx concentration is obtained by calculating the NOx concentration output through use of the “second pumping current−NOx concentration output relational expression” and by multiplying the NOx concentration output by the correction coefficient, rather than multiplying the second pumping current Ip2 (detected by the NOx detection section) directly by the correction coefficient. However, the present inventions is not limited to such a form. For example, the NOx concentration may be obtained at one time by multiplying the IP2 value directly by the correction coefficient and a predetermined value for conversion to the NOx concentration.

Next, the function of a single component in the above-described embodiments may be distributed to a plurality of components, or functions of a plurality of components may be realized by one component. Part of the configuration in each of the above-described embodiments may be omitted. At least part of the configuration in each of the above-described embodiments may be added to the configuration of another embodiment or may replace the configuration of another embodiment. Any embodiments included in the technical ideas specified by the wording of the claims are embodiments of the present disclosure.

The present disclosure may be realized in various forms other than the above-described microcomputer 190. For example, the present disclosure may be realized as a system including the microcomputer 190 as a component, a program that causes a computer to function as the microcomputer 190, a non-transitory tangible recording medium, e.g., a semiconductor memory, in which the program is stored, and a concentration computation method.

### DESCRIPTION OF SYMBOLS

1 . . . multi-gas detection apparatus, 2 . . . multi-gas sensor, 3 . . . control section, 5 . . . sensor element section, 101 . . . NOx detection section, 102 . . . first ammonia detection section, 103 . . . second ammonia detection section, 190 . . . microcomputer, 191 . . . CPU, 192 . . . ROM, 193 . . . RAM.

1. A concentration calculation apparatus comprising:
a nitrogen oxide concentration computation section which calculates a concentration of nitrogen oxide contained in a target gas, wherein
the concentration calculation apparatus is adapted to be connected to a first detection section that outputs a first detection value that changes with the concentration of nitrogen oxide, and to a second detection section that outputs a second detection value that changes with a concentration of ammonia contained in the target gas,
the nitrogen oxide concentration computation section computes the concentration of the nitrogen oxide based on the first detection value, and
the nitrogen oxide concentration computation section computes the concentration of the nitrogen oxide from a value obtained by multiplying the first detection value by a correction coefficient set based on the second detection value.

2. The concentration calculation apparatus according to claim 1, wherein
the nitrogen oxide concentration computation section computes the concentration of the nitrogen oxide from a value obtained by multiplying the first detection value by the correction coefficient and adding a correction addition value to a result of the multiplication, and
the correction addition value is set based on at least the second detection value.

3. A concentration calculation apparatus according to claim 1, further comprising an ammonia concentration computation section that computes the concentration of the ammonia based on the second detection value.

4. The concentration calculation apparatus according to claim 1, wherein the nitrogen dioxide concentration computation section computes the concentration of the nitrogen dioxide based on the second detection value, wherein
the second detection section outputs the second detection value, which changes with the concentration of ammonia contained in the target gas as well as the concentration of nitrogen dioxide contained in the target gas.

5. concentration calculation apparatus according to claim 1, wherein the first detection section and the second detection section are integrally formed to provide a multi-gas sensor.

6. A concentration calculation apparatus comprising:
a nitrogen oxide concentration computation section which computes a concentration of the nitrogen oxide in a target gas; and
an ammonia concentration computation section which computes a concentration of the ammonia in a target gas, wherein
the concentration calculation apparatus is adapted to be connected to a first detection section that outputs a first detection value, which changes with the concentration of nitrogen oxide, and is adapted to be connected to a second detection section that outputs a second detection value, which changes with the concentration of ammonia,
the nitrogen oxide concentration computation section computes the concentration of the nitrogen oxide based the first detection value
the ammonia concentration computation section computes the concentration of the ammonia based the second detection value,
the nitrogen oxide concentration computation section computes the concentration of the nitrogen oxide from a value obtained by multiplying the first detection value by a correction coefficient, said correction coefficient varying in accordance with at least the concentration of the ammonia.

7. A gas detection apparatus comprising:
a first detection section that outputs a first detection value, which changes with a concentration of nitrogen oxide contained in a target gas;
a second detection section that outputs a second detection value, which changes with a concentration of ammonia contained in the target gas; and
the concentration calculation apparatus according to claim 1.

8. A gas detection apparatus comprising:
a first detection section that outputs a first detection value, which changes with a concentration of nitrogen oxide contained in a target gas;
a second detection section that outputs a second detection value, which changes with a concentration of ammonia contained in the target gas; and
the concentration calculation apparatus according to claim 2.
